(19) United States
(12) Patent Application Publication
(10) Pub. No.: US 2025/0189487 A1
(43) Pub. Date: Jun. 12, 2025

(54) EDDY CURRENT (EC) INSPECTION CONFIGURATION SYSTEM AND TECHNIQUE

(71) Applicant: Evident Canada, Inc., Québec (CA)

(72) Inventors: Sylvain Beaulieu, Québec (CA); Jamal Belabed, Québec (CA); Francis Dionne, Donnacona (CA)

(21) Appl. No.: 18/840,341

(22) PCT Filed: Mar. 1, 2023
(86) PCT No.: PCT/CA2023/050261

§ 371 Date: Aug. 21, 2024

Related U.S. Application Data
(60) Provisional application No. 63/268,784, filed on Mar. 2, 2022.

(51) Int. Cl. G01N 27/904; G01N 33/2045

(52) U.S. Cl. CPC G01N 27/904; G01N 33/2045

(57) ABSTRACT

Various approaches can be used for performing eddy current inspection of a structure. Sensor configurations described herein can include flex circuits comprising multiple EC sensor elements. The flex circuit can conform to a region of a structure under test, such as a desired portion of a profile, and such as supported by spacers to maintain a desired stand-off distance between the object under test and the probe assembly. Techniques herein can be used to establish inspection configuration data defining activation or deactivation of respective EC sensors in a probe assembly. For example, a graphical user interface (GUI) can be used to provide graphical feedback concerning one or more attributes of testing, such as indicia of a test probe location or other attributes.

### CLAIM OF PRIORITY

This patent application claims the benefit of priority of Beaulieu et al., U.S. Provisional Patent Application No. 63/268,784, titled “EDDY CURRENT (EC) PROBE CONFIGURATION, TECHNIQUES FOR EC TESTING, AND EC TESTING USER INTERFACE,” filed on Mar. 2, 2022 (Attorney Docket No. 6409.230PRV), which is hereby incorporated by reference herein in its entirety.

### FIELD OF THE DISCLOSURE

This document pertains generally, but not by way of limitation, to apparatus and techniques for non-destructive inspection such as facilitating eddy current inspection, and more particularly, to apparatus and techniques for performing eddy current inspection including establishing configurations for enabling or disabling respective eddy current sensor elements in an eddy current probe assembly.

### BACKGROUND

Non-destructive testing (NDT) can refer to use of one or more different techniques to inspect regions on or within an object, such as to ascertain whether flaws or defects exist, or to otherwise characterize the object being inspected. Examples of non-destructive test approaches can include use of an eddy current testing approach where electromagnetic energy is applied to the object and resulting induced currents on or within the object create electrical signatures that can be detected. For example, values of a detected current (or a related impedance) can provide an indication of the structure of the object under test, such as to indicate a presence of a crack, void, porosity, or other inhomogeneity, such as at or near a surface of a conductive object under test. Eddy current testing can be used as a surface inspection technique for steel structures, such as paired with other inspection techniques (e.g., acoustic inspection) to achieve surface coverage or sub-surface coverage.

### SUMMARY OF THE DISCLOSURE

Eddy current (EC) testing can be used as a non-destructive inspection technique, such as supporting inspection operations during or after manufacturing of an article. For example, steel structures such as a railway rails can be inspected as a portion of a production or acceptance process, such as using an eddy current technique or a combination of eddy current and other inspection modalities such as visual inspection or acoustic inspection. In one approach, an inspection can be performed by a technician using a pencil probe or other probe configuration. Such an approach can present various challenges. For example, eddy current inspection generally involves maintaining a desired spatial relationship between an eddy current sensor and a surface of an object under test. If the sensor is lifted away from the surface of the object under test, test coverage can be impacted and re-scanning may be required. Also, scanning a structure by hand using a raster or other pattern can be time consuming and may lack consistency. Automation such as using fixtures housing an EC probe assembly can assist in improving inspection consistency, but such fixtures are still sensitive to probe misorientation or incorrect probe location relative to the object being inspected.

The present inventors have recognized, among other things, that EC inspection can be facilitated by use of machine-implemented tools (e.g., computer-implemented tools such as providing a user interface or operator interface) to plan and execute EC inspection. An array of eddy current sensors can be used, such as to enhance test productivity by providing greater coverage for each scan or pass. As an illustration, such machine-implemented tools can include or use models representative of respective eddy current array (ECA) probe configurations and related models of respective objects to be inspected. For example, the present subject matter can include or use such models to assist a user in one or more of (a) establishing a specified probe location relative to an object under test for a specified inspection configuration, (b) selecting or deselected respective ones of ECA probe sensors for the specified inspection configuration, or (c) storing the specified inspection configuration for use in controlling a EC inspection operation.

The apparatus and techniques described herein can be used for performing “offline” inspection planning for a future inspection operation, or in an “online” manner where such apparatus and techniques can be used to configure and trigger such inspection. The apparatus and techniques described herein can also be used to facilitate review of inspection results, such as providing a visual representation of EC inspection operation findings overlaid on a representation of the object under test for purposes of review, reporting, or archival. The apparatus and techniques described herein can include use of ECA probe assemblies having a flexible substrate, such as respective probe assemblies configured for inspection of portions of objects under test having complex profiles. As an illustration, the apparatus and techniques described herein can be used to facilitate EC inspection of railway rails, such as to support contemporaneous inspection of a railway rail using multiple ECA inspection probes. For example, such inspection using a configuration technique as described herein can provide coverage of multiple portions of a rail profile using multiple ECA inspection probe assemblies, in a single pass.

In an example, a technique such as a machine-implemented method can support eddy current (EC) inspection, the machine-implemented method comprising receiving a model defining a contour of an object under test, receiving a model of an eddy current array (ECA) probe, the model defining spatial locations of a plurality of eddy current sensors, receiving an indication of the location of the ECA probe relative to the location of the object under test, and in response, indicating respective ones of eddy current sensors amongst the plurality of eddy current sensors to activate, using the received model defining the contour of the object under test, the received model of the ECA probe, and the received indication of the location of the ECA probe. The machine-implemented method can include generating a presentation for a user identifying the indicated respective ones of eddy current sensors to activate. The machine-implemented method can include that the received model defining the ECA probe defines a plurality of spacers, the plurality of spacers establishing a specified stand-off distance between the plurality of eddy current sensors and the object under test when respective ones of the plurality of spacers are in contact with the object under test. For example, the machine-implemented method can include generating a presentation for a user indicating the location of the ECA probe including a location of at least one of the spacers amongst the plurality of spacers and whether the at least one of the spacers amongst the plurality of spacers is within a specified locus.

In an example, a system can support eddy current (EC) inspection, the system comprising processor circuit, a display communicatively coupled with the processor circuit, a user input communicatively coupled with the processor circuit, and a memory circuit comprising instructions that, when executed by the processor circuit cause the processor circuit to receive a model defining a contour of an object under test, receive a model of an eddy current array (ECA) probe, the model defining spatial locations of a plurality of eddy current sensors, receive, using the user input, an indication of the location of the ECA probe relative to the location of the object under test, and in response, generate a presentation for the display indicating respective ones of eddy current sensors amongst the plurality of eddy current sensors to activate, using the received model defining the contour of the object under test, the received model of the ECA probe, and the received indication of the location of the ECA probe. The instructions can include instructions to store an EC inspection configuration comprising data indicative of the respective ones of eddy current sensors to activate and comprising data indicative of the location of the ECA probe relative to the location of the object under test. The instructions can also include instructions to store multiple EC inspection configurations corresponding to respective ECA probe definitions and corresponding locations.

This summary is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates generally an example comprising a non-destructive inspection system, such as can be used to perform at least a portion one or more techniques as shown and described herein.

FIG. 2A illustrates generally an example comprising an eddy current array (ECA) probe assembly, such as having a curved shape.

FIG. 2B illustrates generally an example comprising an eddy current array (ECA) probe assembly comprising respective spacers, such as to maintain a specified stand-off distance between the ECA probe assembly and an object under test.

FIG. 2C illustrates generally an example comprising an eddy current array (ECA) probe assembly included as a portion of an inspection fixture, the inspection fixture having respective actuators.

FIG. 3 illustrates generally an illustrative example of a presentation (e.g., a graphical user interface) that can be presented to a user to assist in establishing an EC inspection configuration.

FIG. 4A and FIG. 4B illustrate generally respective illustrative examples of techniques can be used to provide an indication of respective EC sensors to activate, based on different criteria.

FIG. 5 illustrates generally an illustrative example of a presentation (e.g., a graphical user interface) that can be presented to a user, the illustrative example of FIG. 5 defining a probe selection context.

FIG. 6A illustrates generally a further illustrative example of a presentation (e.g., a graphical user interface) that can be presented to a user, the illustrative example of FIG. 6A defining a probe location context.

FIG. 6B illustrates generally a further illustrative example of a presentation (e.g., a graphical user interface) that can be presented to a user, the illustrative example of FIG. 6B defining a further probe location context showing a magnified (e.g., “zoomed”) view.

FIG. 6C illustrates generally a further illustrative example of a presentation (e.g., a graphical user interface) that can be presented to a user, the illustrative example of FIG. 6C defining an inspection zone context.

FIG. 6D illustrates generally a further illustrative example of a presentation (e.g., a graphical user interface) that can be presented to a user, the illustrative example of FIG. 6D defining an overall EC inspection configuration, such as facilitating respective inspection operations using different EC probe assemblies.

FIG. 7 illustrates generally a further illustrative example of a presentation (e.g., a graphical user interface) that can be presented to a user, the illustrative example of FIG. 7 defining an inspection result reporting context.

FIG. 8 illustrates generally a technique, such as a machine-implemented method, that can include indicating respective ones of eddy current sensors to activate in support of an inspection operation, such as using received models.

FIG. 9 illustrates a block diagram of an example comprising a machine 900 upon which any one or more of the techniques (e.g., methodologies) discussed herein may be performed.

### DETAILED DESCRIPTION

Eddy current (EC) testing can be used as a non-destructive inspection technique, such as supporting inspection operations during or after manufacturing of an article. The system and techniques described in this document can facilitate such testing, such as providing a graphical user interface (GUI) and related machine-implemented tools to aid a user in establishing EC inspection configurations in support of EC testing. Such configurations can be defined as stored data structures providing details concerning probe selection, object under test, probe location, and respective sensors to be activated or deactivated for a corresponding inspection operation. The present subject matter can also include a flaw visualization tool, such as providing indicia of detected flaws overlaid on a visualization of at least a portion of the object under test. Such visualization can be facilitated using the inspection configuration data corresponding to an inspection operation, because such inspection configuration data can indicate the probe type, location, or other information allowing flaws to be localized on the object under test.

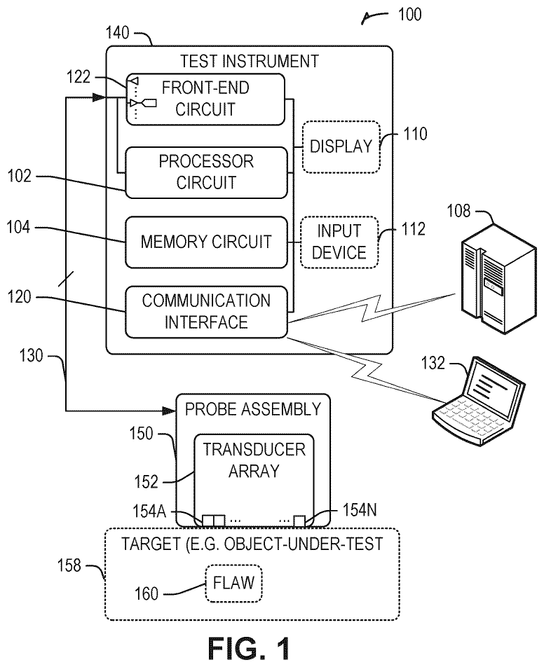

FIG. 1 illustrates generally an example comprising a non-destructive inspection system 100, such as can be used to perform at least a portion one or more techniques as shown and described herein. The non-destructive inspection system 100 can include a test instrument 140, such as a hand-held or portable assembly. The test instrument 140 can be electrically coupled to a probe assembly 150, such as using a multi-conductor interconnect 130. The probe assembly 150 can include one or more transducers, such as an eddy current (EC) transducer array 152 including respective EC sensors 154A through 154N. The transducers array 152 can follow a linear or curved contour or can include an array of elements extending in multiple axes. Element size and pitch can be varied according to the inspection application.

A modular probe assembly 150 configuration can be used, such as to allow a test instrument 140 to be used with various different probe assemblies. Generally, the transducer array 152 includes EC coils, such as located on or within a substrate. The EC coils are electromagnetically coupled with a target 158 (e.g., a test specimen or “object-under-test”). The test instrument 140 can include digital and analog circuitry, such as a front-end circuit 122 including one or more transmit signal chains, receive signal chains, or switching circuitry (e.g., transmit/receive switching circuitry). The transmit signal chain can include amplifier and filter circuitry, such as to provide transmit pulses for delivery through an interconnect 130 to a probe assembly 150. A flaw 160 associated with the target 158 can be detected such as by monitoring an impedance or other electrical characteristic associated with respective sensors 154A through 154N in the transducer array 152.

While FIG. 1 shows a single probe assembly 150 and a single transducer array 152, other configurations can be used, such as multiple probe assemblies connected to a single test instrument 140, or multiple transducer arrays 152 used with a single probe assembly 150. Similarly, a test protocol can be performed using coordination between multiple test instruments 140, such as in response to an overall test scheme established from a respective test instrument 140 or established by another remote system such as a compute facility 108 or general-purpose computing device such as a laptop 132, tablet, smart-phone, desktop computer, or the like. The test scheme may be established according to a published standard or regulatory requirement and may be performed upon initial fabrication or on a recurring basis for ongoing surveillance, as illustrative examples. Generally, as described elsewhere herein, an EC inspection configuration can be established separately, such as executed by a test instrument 140 in a fully automated or semi-automated manner.

The front-end circuit 122 can be coupled to and controlled by one or more processor circuits, such as a processor circuit 102 included as a portion of the test instrument 140. The processor circuit can be coupled to a memory circuit 104, such as to execute instructions that cause the test instrument 140 to perform one or more of EC inspection, processing, or storage of data relating to an EC inspection, or to otherwise perform techniques as shown and described herein. The test instrument 140 can be communicatively coupled to other portions of the system 100, such as using a wired or wireless communication interface 120.

For example, performance of one or more techniques as shown and described herein can be accomplished on-board the test instrument 140 or using other processing or storage facilities such as using a compute facility 108 or a general-purpose computing device such as a laptop 132, tablet, smart-phone, desktop computer, or the like. For example, processing tasks that would be undesirably slow if performed on-board the test instrument 140 or beyond the capabilities of the test instrument 140 can be performed remotely (e.g., on a separate system), such as in response to a request from the test instrument 140. The test instrument 140 can include a display 110, such as for presentation of configuration information or results, and an input device 112 such as including one or more of a keyboard, trackball, function keys or soft keys, mouse-interface, touch-screen, stylus, or the like, for receiving operator commands, configuration information, or responses to queries.

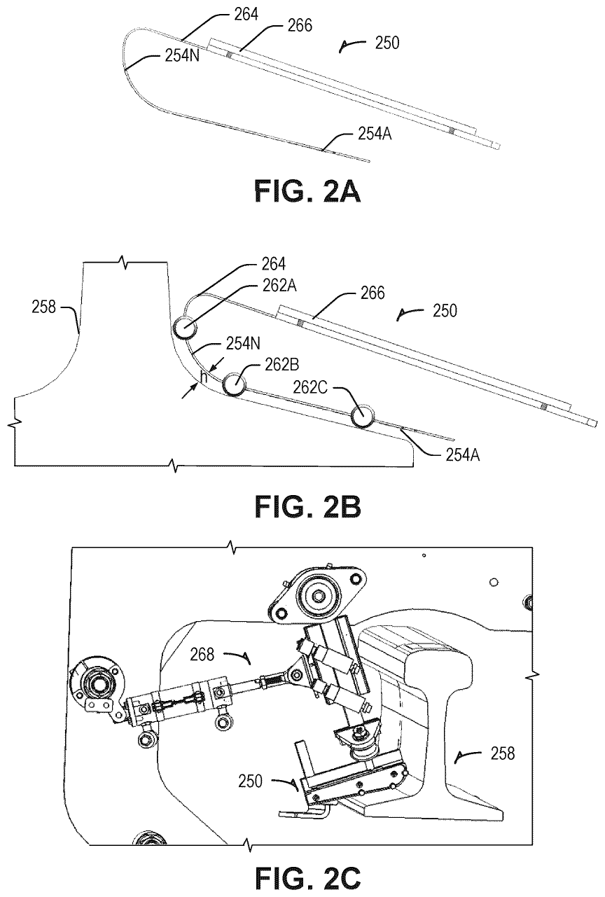

FIG. 2A illustrates generally an example comprising an eddy current array (ECA) probe assembly 250, such as having a curved shape. The probe assembly 250 can include a rigid portion 266, such as can include an interconnect for coupling electrical conductors in the probe assembly 250 to other test instrumentation. The probe assembly 250 can include a flexible substrate 264, such as having a curved profile. The flexible substrate 264 can include or support respective eddy current sensors (e.g., an EC sensor 254A through an EC sensor 254N). For example, the probe assembly 250 can include a linear array of EC sensors, such as including 16 coils. Other fixturing, such as a rigid core, can provide a shape to which the flexible substrate 264 can conform. In this manner, a common flexible probe configuration can be used across multiple different profiles by bending or curving the flexible substrate 264 accordingly.

FIG. 2B illustrates generally an example comprising an eddy current array (ECA) probe assembly 250 comprising respective spacers, such as to maintain a specified stand-off distance between the ECA probe assembly and an object under test. Similar to the example of the probe assembly 250 of FIG. 2A, the probe assembly 250 of FIG. 2B includes a rigid portion 266, a flexible substrate 264, and sensors such as an EC sensor 254A through an EC sensor 254N). A first spacer 262A, a second spacer 262B, and a third spacer 262C can maintain a specified stand-off distance between the probe assembly 250 and an object under test 258 (e.g., the foot of a railway rail in the example of FIG. 2B). As an illustration, the spacers (first spacer 262A, second spacer 262B, and third spacer 262C) can include a carbide material, such as defining carbide pins or cylindrical structures. In this manner, the first spacer 262A, second spacer 262B, and third spacer 262C can be abrasion and damage resistant, such as protecting the probe assembly 250 EC sensor 254A through EC sensor 254N from damage, and maintaining a specified distance, “h,” between the flexible substrate 264, with EC sensor 254A through an EC sensor 254N, and the object under test 258.

FIG. 2C illustrates generally an example comprising an eddy current array (ECA) probe assembly 250 included as a portion of an inspection fixture 268, the inspection fixture having respective actuators. As mentioned above, a flexible portion of the probe assembly 250 can be mounted in the inspection fixture 268. The inspection fixture 268 can be robotically manipulated to be translated along an object under test 258, or as shown illustratively in FIG. 2C, the inspection fixture 268 can be supported by or incorporated in a frame assembly, with the object under test 258 being translated relative to the probe assembly 250 and inspection fixture 268. For example, as shown illustratively in FIG. 2C, the object under test 258 can include a railway rail, and the rail can be conveyed past the inspection fixture 268.

As shown and described elsewhere herein, a location of the probe assembly 250 can be defined by a stored inspection configuration, and sensors in the probe assembly can be activated or deactivated according to the stored configuration. The probe assembly 250 can be positioned robotically, or using actuators, to establish the probe assembly 250 at the specified stored location corresponding to the stored inspection configuration. For example, as shown in FIG. 2C, a combination of two pivoting elements and two linear actuators can be used to provide various degrees of freedom for probe positioning. Such examples are merely illustrative and other configurations can be used. Multiple probe assemblies 250 can be positioned either in a gang or independently, such as using respective fixtures 268, to achieve coverage of different regions of the object under test 258. In this manner, multiple inspection operations can be performed contemporaneously as the object under test 258 is translated relative to the respective fixtures 268. Such inspection operations can include longitudinal, transverse, and oblique probe configurations, relative to a long axis of the object under test 258.

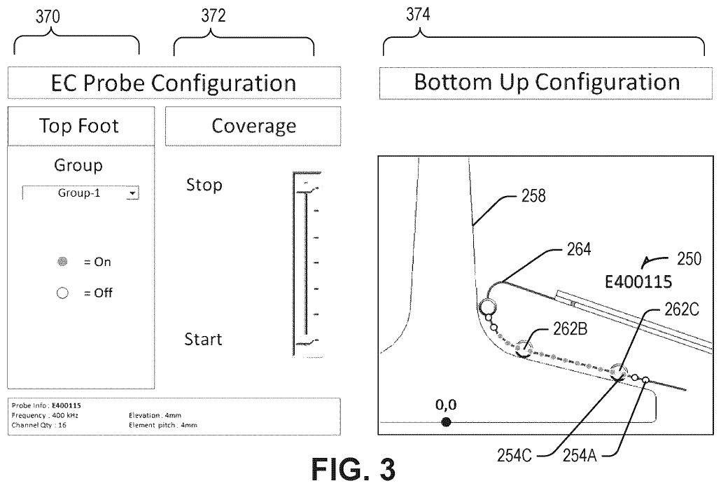

FIG. 3 illustrates generally an illustrative example of a presentation (e.g., a graphical user interface) that can be presented to a user to assist in establishing an EC inspection configuration. FIG. 3 illustrates conceptually various aspects of an inspection configuration that can be established, retrieved, or stored, aided by a machine-implemented technique and a graphical presentation to a user. For example, in the probe position display 374, a probe assembly 250 can be shown, such as corresponding to a selected probe (“E400115”). The probe assembly 250 representation can be defined by a model comprising a point cloud including locations of the flexible substrate 264, spacers 262A, 262B, and 262C, and respective EC sensors such as a sensor 254A and a sensor 254C. The object under test 258 can also be defined by a model comprising a point cloud, such as defining a coordinate system with an origin (0,0) as shown illustratively in the probe position display 374. Test coverage along a longitudinal extent of the object under test 258 can be controlled, such as using the coverage control region 372, and a group of EC sensors to be activated or deactivated can be controlled using the sensor configuration region 370. For example, as shown and described elsewhere below, a machine-implemented technique can provide an indication (e.g., a suggestion or selection) of respective sensors to be activated or deactivated, based on various criteria. For example, such an indication can include shading, texturing, or coloring of respective sensor elements as shown illustratively in FIG. 3 (with sensors indicated as active for the inspection operation being shaded, and sensors indicated as inactive being non-shaded). For example, EC sensor 254A is non-shaded, and EC sensor 254C is shaded. Generally, if the inspection operation is stored, retrieved, and then executed, the EC inspection can be carried out using the indicated active sensors. Depending on the configuration of the machine-implemented tool, automated selection of sensors can be a suggestion for the user, with the user able to override automatically-determined active and inactive sensor assignments.

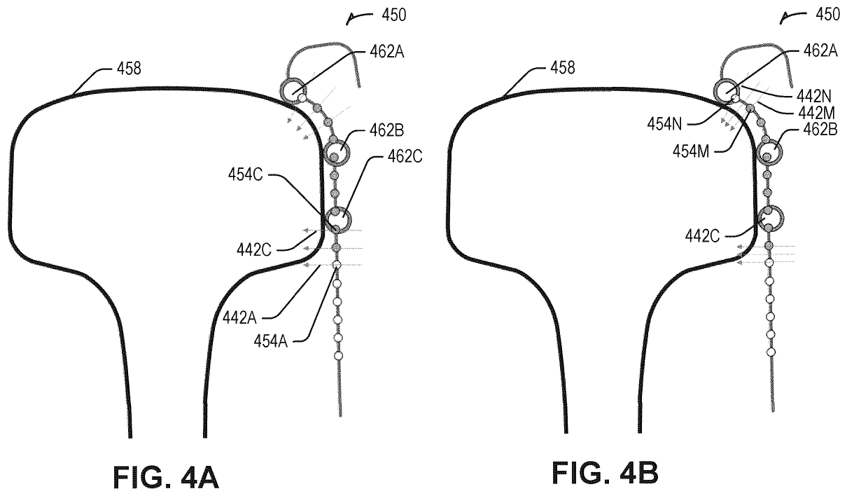

FIG. 4A and FIG. 4B illustrate generally respective illustrative examples of techniques can be used to provide an indication of respective EC sensors to activate, based on different criteria. As mentioned above, an indication of respective ones of EC sensors to be activated can be automated or semi-automated. For example, as shown in FIG. 4A and FIG. 4B, a model such as point cloud or other data indicative of a geometry of an object under test 458 (in this example a head of a railway rail) can be received, such as retrieved from a data file. In FIG. 4A and FIG. 4B, a model of a test probe assembly 450 can also be received, such as retrieved from a data file. For example, a user can select an object under test 458 for an inspection operation, and a corresponding probe assembly 450 to be used. A location of the probe assembly 450 can be displayed, and the user can manipulate the probe location of the probe assembly 450. As discussed below, an indication can be provided indicative of whether the probe assembly 450 location is nominal, such as by changing a color, texture, or shading of an element in a graphical user interface. For example, such visual indicia can be presented in relation to a location of one or more spacers 462A, 462B, or 462C.

A respective EC sensor 454A in the ECA of the probe assembly 450 can have an associated normal vector 442A (e.g., defined as perpendicular to a tangent defined at a location along the substrate of the probe assembly 450, such as extending outward from a plane defined by a correspond EC sensor or a substrate). If the normal vector 442A fails to intersect a contour of the object under test 458 within a specified distance of the probe assembly 450, the respective EC sensor 454A can be deactivated (or an indication of such deactivation can be presented as a suggested EC sensor configuration). Similarly, if a respective EC sensor 454C has a corresponding normal vector 442C that intersects the contour of the object under test 458, then the sensor can be activated (or an indication of such activation can be presented as a suggested EC sensor configuration). In this manner, generic probe assemblies can be used for different object under test 458 geometries, with activation or deactivation of respective sensors performed to provide a nominal inspection configuration, such as suppressing acquisition of inspection data from particular sensors that are spaced apart or lifted off a surface of the object under test 458 due to the probe assembly 450 geometry extending beyond a contour of the object under test 458. As an illustrative example, sensors can be indicated as activated if a distance between the sensor and a contour of the object under test is less than three millimeters as indicated by a corresponding normal vector (e.g., three millimeters can be a lift-off limit or detection limit for the probe assembly 450).

Other criteria can be used to provide indications of EC sensors to be activated or deactivated. For example, referring to FIG. 4B, the probe assembly 450, spacers 462A, 462B, and 462C, and object under test 458 can be in the same locations relative to each other as in FIG. 4A. In FIG. 4B, normal vectors can be determined in a manner similar to FIG. 4A, such as a normal vector 442M corresponding to an EC sensor 454M and a normal vector 442N corresponding to an EC sensor 454N. An indication of a degree of curvature can be determined such as by determining a distance between the respective EC sensor 454M or 454N and a contour of the object under test 458, such as using the corresponding normal vectors 442M and 442M. As an illustration, a radius of curvature or other value can be determined, such as to be compared against a specified threshold. For example, if such determination indicates a relatively sharp radius of curvature, one or more EC sensors located at or nearby such a curvature can be indicated as deactivated. As shown in the illustration of FIG. 4B, the EC sensor 454N is shown as non-shaded, and therefore is indicated to be deactivated, even though the EC sensor 454N would meet the criterion for activation according to the technique of FIG. 4A. This deactivation indication in FIG. 4B is because the EC sensor 454N is in a region of relative sharp curvature.

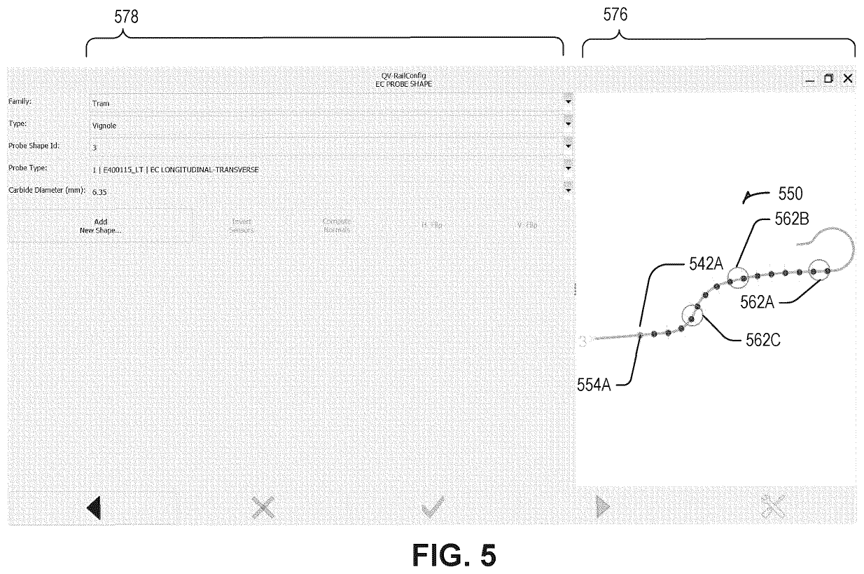

FIG. 5 illustrates generally an illustrative example of a presentation (e.g., a graphical user interface) that can be presented to a user, the illustrative example of FIG. 5 defining a probe selection context. In the example of FIG. 5, an ECA probe assembly 550 can be selected by a user, such as using a probe and object under test selection pane 578. As shown in the illustration of FIG. 5, a machine-implemented method can receive an ECA probe assembly 550 definition, such as retrieved in response to a user selection. Other aspects of the inspection configuration can also be input by a user, such as a selection of an object under test, an inspection type, or other information such as spacer (e.g., “carbide”) pin size, associated with the ECA probe assembly 550 and related inspection fixture. Similar to other examples described in this document, a probe display pane 576 can show a representation of the received model of the ECA probe assembly 550, including the probe substrate shape, locations of spacers 562A, 562B, and 562C, and individual EC sensors such as an EC sensor 554A and corresponding normal vector 542A. A simplified probe shape identifier (“3”) can also be displayed as shown illustratively in FIG. 5.

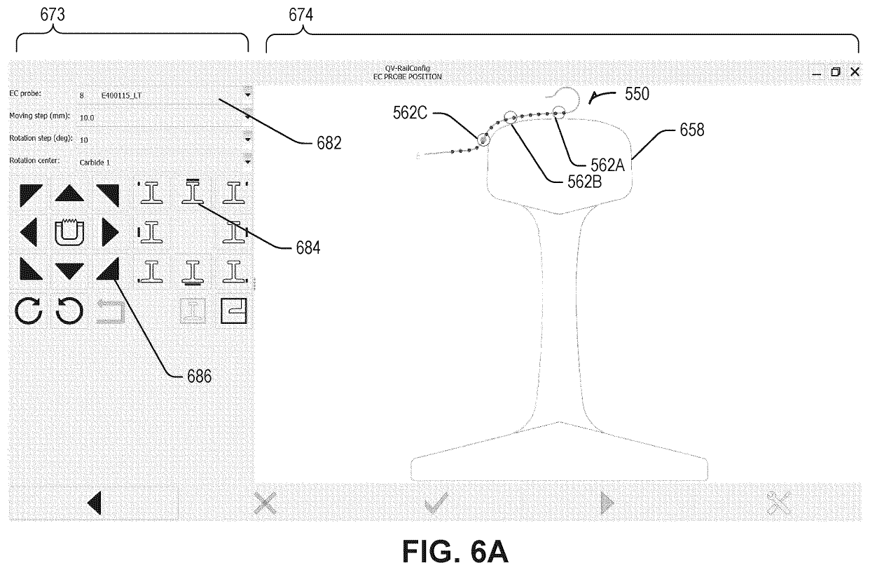

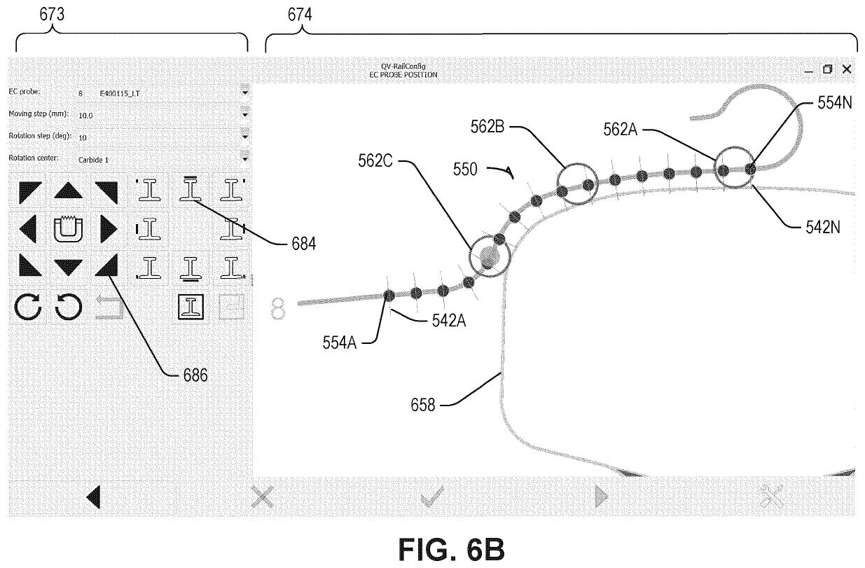

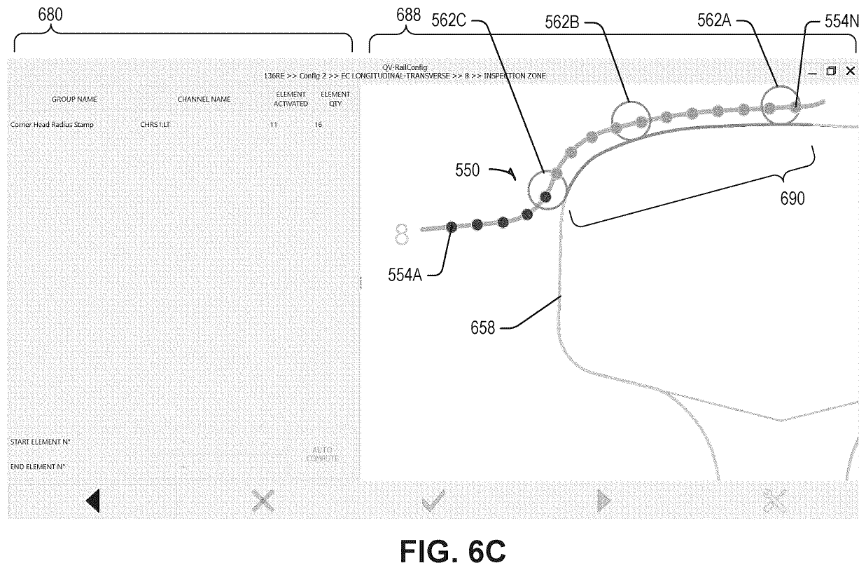

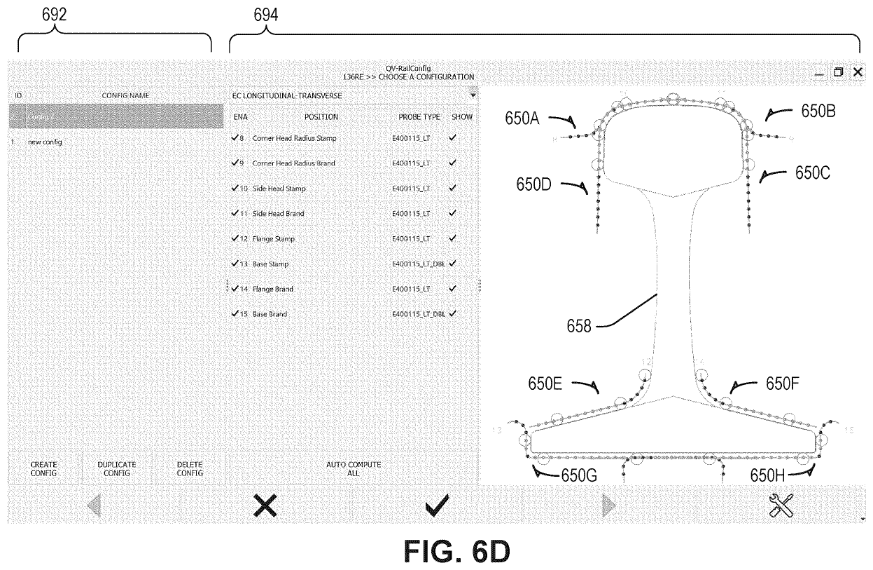

FIG. 6A illustrates generally a further illustrative example of a presentation (e.g., a graphical user interface) that can be presented to a user, the illustrative example of FIG. 6A defining a probe location context. Similar to other examples herein, such as shown in FIG. 5, a location of an ECA probe assembly 550 can be displayed, such as relative to a model of an object under test 658. A probe position display 674 can be presented in a pane, such as along with a configuration and position control pane 673. For example, a probe can be selected at 682, and a machine-implemented method can receive and display a representation of a model of an ECA probe assembly 550. The probe position relative to the object under test 658, such as using translation controls at 686 or by rotating the probe representation. Quick shortcut inputs can be provided such as shown at 684, to roughly position a representation of the ECA probe assembly 550, with the translation controls used for fine tuning. Locations of spacers 562A, 562B, and 562C can be shown. When a respective one (or more) of the spacers 562A, 562B, or 562C is within a specified locus, a representation in the probe position display 674 can indicate that the respective one (or more) of the spacers 562A, 562B, or 562C is in a nominal or otherwise specified location for inspection. For example, because EC inspection is sensitive to a distance between respective EC sensors in the ECA probe assembly 550 and the object under test 658, a visual aid can assist a user in establishing an appropriate probe position for inspection. As an illustration, circles representing the spacers 562A, 562B, and 562C can automatically change color, texture, or shape, as illustrative examples, to indicate whether a respective one (or more) of the spacers 562A, 562B, and 562C is in an appropriate location for inspection.

FIG. 6B illustrates generally a further illustrative example of a presentation (e.g., a graphical user interface) that can be presented to a user, the illustrative example of FIG. 6B defining a further probe location context showing a magnified (e.g., “zoomed”) view. Similar to the example of FIG. 6A, in the magnified view of FIG. 6B, the ECA probe assembly 550 location or orientation (or both) can be manipulated (such as having greater precision than the view of FIG. 6A). The example of FIG. 6B includes a probe position display 674, configuration and position control pane 673, representation of model of ECA probe assembly 550 relative to representation of model of object under test 658, spacers 562A, 562B, 562C, shortcut input at 684, translation input at 686. The ECA probe assembly 550 representation can include respective representations of vectors normal to respective EC sensors, as shown and described elsewhere herein. For example, as shown in FIG. 6B, a respective EC sensor 554A can have corresponding vectors extending normally outward, such as a normal vector 542A, and a respective EC sensor 554N can have corresponding vectors extending normally outward, such as a normal vector 542N. Vectors can extend in opposite directions as shown in FIG. 6B, because the ECA probe assembly 550 may be used in another orientation where an opposite surface of the substrate is facing the object under test 658. As in the example of FIG. 6A and other examples herein, an indication of probe location or alignment can be provided such as by shading, texturing, or coloring an indicium in the probe position display 674. For example, a respective one (or more) of spacers 562A, 562B, or 562C can change color when the ECA probe assembly 550 is in a proper orientation for an inspection operation. The location of the ECA probe assembly 550 can be stored, such as to facilitate a later inspection operation or to correlate and display received EC inspection results with spatial locations of faults based on the stored ECA probe assembly 550 location relative to the object under test 658.

FIG. 6C illustrates generally a further illustrative example of a presentation (e.g., a graphical user interface) that can be presented to a user, the illustrative example of FIG. 6C defining an inspection zone context. A user can select and manipulate a representation of ECA probe assembly 550 as shown and described elsewhere, such as discussed above in relation to FIG. 6A and FIG. 6B. In the inspection zone context, the established ECA probe assembly 550 location can be used, along with the normal vectors or other criteria to provide an indication of respective EC sensors to be activated or deactivated for an inspection operation. For example, a group of EC sensors can be assigned an identifier in a sensor group pane 680, and an inspection zone pane 688 can be provided to display an inspection zone 690 (e.g., a region of surface coverage for an EC inspection operation). Visual indicia can be provided for respective ones of the EC sensors in the ECA probe assembly 550 to be activated or deactivated, such as based on normal vector and distance between a respective sensor and a contour of the object under test 658. For example, in the illustration of FIG. 6C, an EC sensor 554A is indicated as deactivated (and is far away from the object under test 658 relative to other sensors). By contrast, a sensor 554N is indicated as active. A user can accept the suggested probe activation configuration provided automatically, or, in an example, the user can override the automated indications and manually activate or deactivate EC sensors. Once a sensor configuration and corresponding inspection zone are established, the probe location and selected EC sensors to activate can be stored, such as to facilitate a later inspection operation. Alternatively (or in addition), a user interface similar to the example of FIG. 6C or other examples herein can be provided to facilitate inspection in an “online” manner.

FIG. 6D illustrates generally a further illustrative example of a presentation (e.g., a graphical user interface) that can be presented to a user, the illustrative example of FIG. 6D defining an overall EC inspection configuration, such as facilitating respective inspection operations using different EC probe assemblies. The sequence of selecting a probe, establishing a probe location relative to an object under test, and selecting sensors to be activated can be performed for various different ECA probe assembly configurations. Generally, an EC inspection protocol can include multiple scans corresponding to different sensor configurations and positions to achieve desired inspection coverage. For example, as shown in illustratively in FIG. 6D, an overall inspection configuration can be established or selected by a user in a selection pane 692, and the corresponding selected probe assemblies, probe assembly positions, active EC sensors, or other attributes can be shown graphically in relation to an object under test 658 as shown in in the probe configuration view 694. For example, for a selected inspection configuration, models and corresponding visual representations of eight different probe assemblies 650A, 650B, 650C, 650D, 650E, 650F, 650G, and 650H can be displayed relative to the object under test 658. Probe attributes such as selected EC sensors to be activated or inactive can be displayed as in other examples herein, and a simplified probe number can be displayed to aid in identifying respective probes (such as “8” in relation to a first probe assembly 650A). The configuration shown in the configuration view 694 can correspond to inspection operations to be performed contemporaneously by the different probes mounted in respective fixtures, supporting inspection of all displayed regions along the object under test 658 in as few as a single pass. The configuration selected in the selection pane 692 can be stored, such as retrieved by automated or semi-automated inspection equipment for use in controlling an inspection operation. For example, a user can provide an input selecting a desired inspection configuration, and an inspection operation can be triggered, using the corresponding inspection configuration.

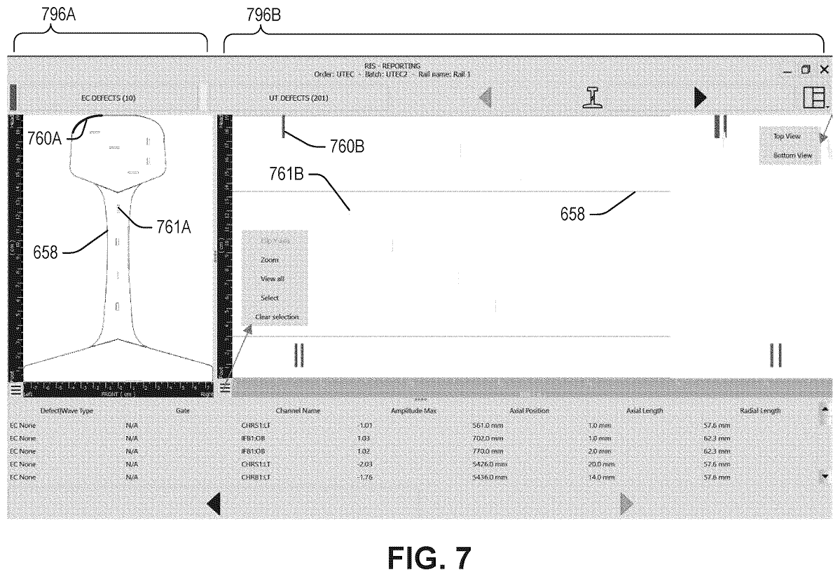

FIG. 7 illustrates generally a further illustrative example of a presentation (e.g., a graphical user interface) that can be presented to a user, the illustrative example of FIG. 7 defining an inspection result reporting context. Stored inspection configuration data, such as established or modified using the other examples herein can also be used to facilitate visualization or reporting of inspection results. For example, because an ECA probe assembly location is established as part of an inspection configuration, the ECA probe assembly and corresponding sensor locations are known relative to a contour of the object under test 658. Accordingly, logged inspection results corresponding to individual sensors, groups of sensors, or probe assemblies can be correlated with such location information to overlay a representation of a flaw, defect, or other feature on a visual representation of the object under test 658. For example, in a first view 796A (e.g., an end view) of the object under test, a first location 760A can be highlighted (e.g., using a color, texture, or shading that provides an indication of a flaw or defect location). In another view 796B, such as an elevational or side view of the object under test 658, a corresponding indication at a second location 760B can be highlighted. In this manner, a potential flaw or defect location can be localized along the actual object under test 658, such as during or after an inspection operation. The visualization of FIG. 7 is not restricted to eddy current inspection data. For example, a result of an acoustic inspection can also be overlaid, such as showing a flaw or defect highlighted at 761A in the first view 796A and at 761B in the second view 796B. Inspection results (e.g., suspected flaw or defect locations) can also be shown in tabular form, as illustrated generally in the bottom pane of FIG. 7.

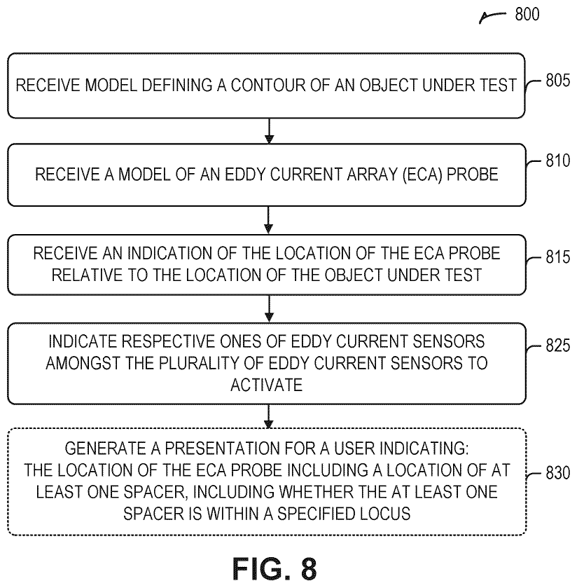

FIG. 8 illustrates generally a technique 800, such as a machine-implemented method, that can include indicating respective ones of eddy current sensors to activate in support of an inspection operation, such as using received models. At 805, a model can be received defining a contour of an object under test. As mentioned above, such a model can include a data structure comprising a point cloud representation, or other representation. At 810, a model of an eddy current array probe can be received, such as including a data structure comprising a point cloud representation, or other representation. The models mentioned above can be selected or established by a user, such as stored, and the machine-implemented method can receive such models by retrieving them from storage, such as in response to a user selection. At 815, an indication can be received of a location of the ECA probe (e.g., a location of the model representing the ECA probe) relative to the object under test (e.g., a model of the object under test, or at least a portion thereof). Receiving such an indication can include retrieving data indicative of the stored location or receiving a user input orienting or positioning a representation of the ECA probe relative to the object under test, such as using a graphical user interface as shown and described in relation to other examples herein. At 825, respective ones of eddy current sensors amongst a plurality of eddy current sensors of the ECA probe can be indicated as active (e.g., indicated as to be activated during a corresponding inspection operation). Such indication can be performed using the received model defining the contour of the object under test, the received model of ECA probe, and the received indication of the location of the ECA probe. For example, such indication can be the result one or more criteria such as using normal vectors, and respective EC sensor distances from the object under test, as described elsewhere herein. Optionally, as shown at 830, a presentation can be generated for a user indicating a location of the ECA probe, including a location of at least one spacer, including whether the at least one spacer is within a specified locus. Such an approach can help a user to guide a probe location to a nominal probe location. A stored location of the ECA probe relative to the object under test can also be used for generating a presentation of an inspection result, such as overlaying a flaw or defect location on a representation of the object under test.

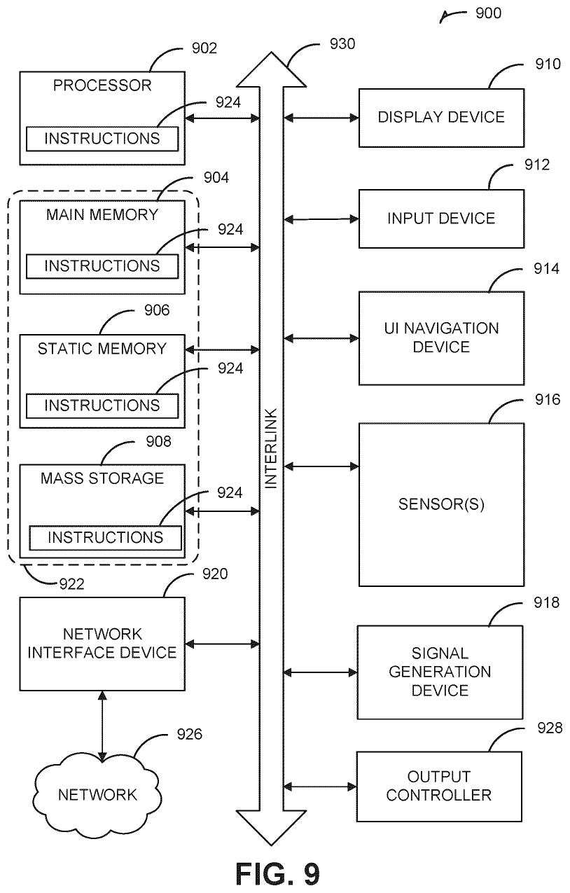

FIG. 9 illustrates a block diagram of an example comprising a machine 900 upon which any one or more of the techniques (e.g., methodologies) discussed herein may be performed. Machine 900 (e.g., computer system) may include a hardware processor 902 (e.g., a central processing unit (CPU), a graphics processing unit (GPU), a hardware processor core, or any combination thereof), a main memory 904 and a static memory 906, connected via an interlink 930 (e.g., link or bus), as some or all of these components may constitute hardware for systems or related implementations discussed above.

Specific examples of main memory 904 include Random Access Memory (RAM), and semiconductor memory devices, which may include storage locations in semiconductors such as registers. Specific examples of static memory 906 include non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; RAM; or optical media such as CD-ROM and DVD-ROM disks.

The machine 900 may further include a display device 910, an input device 912 (e.g., a keyboard), and a user interface (UI) navigation device 914 (e.g., a mouse). In an example, the display device 910, input device 912, and UI navigation device 914 may be a touch-screen display. The machine 900 may include a mass storage device 908 (e.g., drive unit), a signal generation device 918 (e.g., a speaker), a network interface device 920, and one or more sensors 916, such as a global positioning system (GPS) sensor, compass, accelerometer, or some other sensor. The machine 900 may include an output controller 928, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The mass storage device 908 may comprise a machine-readable medium 922 on which is stored one or more sets of data structures or instructions 924 (e.g., software) embodying or utilized by any one or more of the techniques or functions described herein. The instructions 924 may also reside, completely or at least partially, within the main memory 904, within static memory 906, or within the hardware processor 902 during execution thereof by the machine 900. In an example, one or any combination of the hardware processor 902, the main memory 904, the static memory 906, or the mass storage device 908 comprises a machine readable medium.

Specific examples of machine-readable media include, one or more of non-volatile memory, such as semiconductor memory devices (e.g., EPROM or EEPROM) and flash memory devices; magnetic disks, such as internal hard disks and removable disks; magneto-optical disks; RAM; or optical media such as CD-ROM and DVD-ROM disks. While the machine-readable medium is illustrated as a single medium, the term “machine readable medium” may include a single medium or multiple media (e.g., a centralized or distributed database, or associated caches and servers) configured to store the one or more instructions 924.

An apparatus of the machine 900 includes one or more of a hardware processor 902 (e.g., a central processing unit (CPU), a graphics processing unit (GPU), a hardware processor core, or any combination thereof), a main memory 904 and a static memory 906, sensors 916, network interface device 920, antennas, a display device 910, an input device 912, a UI navigation device 914, a mass storage device 908, instructions 924, a signal generation device 918, or an output controller 928. The apparatus may be configured to perform one or more of the methods or operations disclosed herein.

The term “machine readable medium” includes, for example, any medium that is capable of storing, encoding, or carrying instructions for execution by the machine 900 and that cause the machine 900 to perform any one or more of the techniques of the present disclosure or causes another apparatus or system to perform any one or more of the techniques, or that is capable of storing, encoding or carrying data structures used by or associated with such instructions. Non-limiting machine-readable medium examples include solid-state memories, optical media, or magnetic media. Specific examples of machine-readable media include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; Random Access Memory (RAM); or optical media such as CD-ROM and DVD-ROM disks. In some examples, machine readable media includes non-transitory machine-readable media. In some examples, machine readable media includes machine readable media that is not a transitory propagating signal.

The instructions 924 may be transmitted or received, for example, over a communications network 926 using a transmission medium via the network interface device 920 utilizing any one of a number of transfer protocols (e.g., frame relay, internet protocol (IP), transmission control protocol (TCP), user datagram protocol (UDP), hypertext transfer protocol (HTTP), etc.). Example communication networks include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®), IEEE 802.15.4 family of standards, a Long Term Evolution (LTE) 4G or 5G family of standards, a Universal Mobile Telecommunications System (UMTS) family of standards, peer-to-peer (P2P) networks, satellite communication networks, among others.

In an example, the network interface device 920 includes one or more physical jacks (e.g., Ethernet, coaxial, or other interconnection) or one or more antennas to access the communications network 926. In an example, the network interface device 920 includes one or more antennas to wirelessly communicate using at least one of single-input multiple-output (SIMO), multiple-input multiple-output (MIMO), or multiple-input single-output (MISO) techniques. In some examples, the network interface device 920 wirelessly communicates using Multiple User MIMO techniques. The term “transmission medium” shall be taken to include any intangible medium that is capable of storing, encoding or carrying instructions for execution by the machine 900, and includes digital or analog communications signals or other intangible medium to facilitate communication of such software.

### VARIOUS NOTES

Each of the non-limiting aspects above can stand on its own or can be combined in various permutations or combinations with one or more of the other aspects or other subject matter described in this document.

The above detailed description includes references to the accompanying drawings, which form a part of the detailed description. The drawings show, by way of illustration, specific embodiments in which the invention can be practiced. These embodiments are also referred to generally as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc., are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Such instructions can be read and executed by one or more processors to enable performance of operations comprising a method, for example. The instructions are in any suitable form, such as but not limited to source code, compiled code, interpreted code, executable code, static code, dynamic code, and the like. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A machine-implemented method supporting eddy current (EC) inspection, the machine-implemented method comprising:
receiving a model defining a contour of an object under test;
receiving a model of an eddy current array (ECA) probe, the model defining spatial locations of a plurality of eddy current sensors;
receiving an indication of a location of the ECA probe relative to the location of the object under test; and
in response, indicating respective ones of eddy current sensors amongst the plurality of eddy current sensors to activate, using the received model defining the contour of the object under test, the received model of the ECA probe, and the received indication of the location of the ECA probe.

2. The machine-implemented method of claim 1, comprising generating a presentation for a user identifying the indicated respective ones of eddy current sensors to activate.

3. The machine-implemented method of claim 1, wherein the received model defining the ECA probe defines a plurality of spacers, the plurality of spacers establishing a specified stand-off distance between the plurality of eddy current sensors and the object under test when respective ones of the plurality of spacers are in contact with the object under test.

4. The machine-implemented method of claim 3, comprising generating a presentation for a user indicating:
the location of the ECA probe including a location of at least one of the spacers amongst the plurality of spacers; and
whether the at least one of the spacers amongst the plurality of spacers is within a specified locus.

5. The machine-implemented method of claim 1, wherein the indicating the respective ones of eddy current sensors to activate amongst the plurality of eddy current sensors comprises determining an orientation of the respective ones of eddy current sensor relative to the contour of the object under test according to the received model defining the contour of the object under test, the received model of the ECA probe, and the received indication of the location of the ECA probe.

6. The machine-implemented method of claim 5, wherein the indicating the respective ones of eddy current sensors to activate amongst the plurality of eddy current sensors comprises determining respective vectors extending in a direction normal to a plane of the respective ones of eddy current sensors.

7. The machine-implemented method of claim 6, wherein the indicating the respective ones of eddy current sensors to activate amongst the plurality of eddy current sensors comprises indicating other respective ones of the eddy current sensors to deactivate, the other respective ones of the eddy current sensors having respective normal vectors that fail to intersect the contour of the object under test according to the received model defining the contour of the object under test, the received model of the ECA probe, and the received indication of the location of the ECA probe.

8. The machine-implemented method of claim 6, wherein the indicating the respective ones of eddy current sensors to activate amongst the plurality of eddy current sensors comprises indicating other respective ones of the eddy current sensors to deactivate, the other respective ones of the eddy current sensors having respective normal vectors that are associated with a locus along the contour of the object under test having a curvature exceeding a specified threshold according to the received model defining the contour of the object under test, the received model of the ECA probe, and the received indication of the location of the ECA probe.

9. The machine-implemented method of claim 1, comprising triggering an inspection operation by the ECA probe array using the indicated respective ones of eddy current sensors amongst the plurality of eddy current sensors.

10. The machine-implemented method of claim 1, comprising generating a presentation of a result of an inspection operation carried out using the indicated respective ones of eddy current sensors amongst the plurality of eddy current sensors including graphically overlaying an indicium of an inspection result on a representation of the model defining the contour of the object under test.

11. The machine-implemented method of claim 10, comprising presenting at least two different views of a representation of the object under test.

12. The machine-implemented method of claim 1, comprising receiving an indication of a model of an eddy current array (ECA) probe from amongst multiple ECA probe configurations and corresponding models.

13. (canceled)

14. The machine-implemented method of claim 1, wherein the object under test is a railway rail.

15. A system supporting eddy current (EC) inspection, the system comprising:
a processor circuit;
a display communicatively coupled with the processor circuit;
a user input communicatively coupled with the processor circuit; and
a memory circuit comprising instructions that, when executed by the processor circuit cause the processor circuit to:
receive a model defining a contour of an object under test;
receive a model of an eddy current array (ECA) probe, the model defining spatial locations of a plurality of eddy current sensors;
receive, using the user input, an indication of a location of the ECA probe relative to the location of the object under test; and
in response, generate a presentation for the display indicating respective ones of eddy current sensors amongst the plurality of eddy current sensors to activate, using the received model defining the contour of the object under test, the received model of the ECA probe, and the received indication of the location of the ECA probe.

16. The system of claim 15, wherein the instructions comprise instructions to store an EC inspection configuration comprising data indicative of the respective ones of eddy current sensors to activate and comprising data indicative of the location of the ECA probe relative to the location of the object under test.

17. The system of claim 16, wherein the instructions comprise instructions to store multiple EC inspection configurations corresponding to respective ECA probe definitions and corresponding locations.

18. The system of claim 16, comprising at least one ECA probe and corresponding EC inspection instrument;
wherein the EC inspection instrument is configured to use at least one stored EC inspection configuration to perform an EC inspection.

19. (canceled)

20. The system of claim 18, wherein the at least one stored EC inspection configuration comprises a data structure defining a probe identification, probe location, and the respective ones of eddy current sensors amongst the plurality of eddy current sensors to activate.

21. The system of claim 18, wherein the object under test is translated relative to the at least one ECA probe; and
wherein the system comprises at least one actuator to position the at least one ECA probe at the indicated location of the ECA probe relative to the location of the object under test established previously.

22. The system of claim 15, wherein the instructions comprise instructions to generate a presentation of a result of an inspection operation carried out using the indicated respective ones of eddy current sensors amongst the plurality of eddy current sensors including graphically overlaying an indicium of an inspection result on a representation of the model defining the contour of the object under test.

23. The system of claim 15, wherein the object under test is a railway rail.

24. A system supporting eddy current (EC) inspection, the system comprising:
a means for receiving a model defining a contour of an object under test, and a model of an eddy current array (ECA) probe, the model of the ECA probe defining spatial locations of a plurality of eddy current sensors;
a means for receiving an indication of the location of the ECA probe relative to the location of the object under test; and
a means for indicating respective ones of eddy current sensors amongst the plurality of eddy current sensors to activate, using the received model defining the contour of the object under test, the received model of the ECA probe, and the received indication of the location of the ECA probe.

25. (canceled)
